(19) United States
(12) Patent Application Publication
(10) Pub. No.: US 2025/0380964 A1
(43) Pub. Date: Dec. 18, 2025

(54) SYSTEMS, METHODS AND DEVICES FOR PERFORMING ENDOSCOPIC PROCEDURES

(71) Applicant: Endeau, Inc., Escondido, CA (US)

(72) Inventors: Kenneth F. Binmoeller, Rancho Santa Fe, CA (US); Allen Newman, Rancho Santa Fe, CA (US); Matt Yurek, San Diego, CA (US)

(73) Assignee: Endeau, Inc., Escondido, CA (US)

(21) Appl. No.: 19/062,004

(22) Filed: Feb. 24, 2025

Related U.S. Application Data
(63) Continuation of application No. 16/929,057, filed on Jul. 14, 2020.
Application No. 16/929,057 is a continuation-in-part of application No. 16/775,219, filed on Jan. 28, 2020, now abandoned.
Application No. 16/775,219 is a continuation of application No. 14/777,393, filed on Sep. 15, 2015, now Pat. No. 10,542,871.
(60) Provisional application No. 61/801,427, filed on Mar. 15, 2013.
(60) Provisional application No. 61/786,520, filed on Mar. 15, 2013.

(51) Int. Cl. A61B 17/34; A61B 1/00; A61B 1/012; A61B 1/04; A61B 1/31; A61B 17/32; A61B 17/3205

(52) U.S. Cl. CPC A61B 17/3421; A61B 1/00082; A61B 1/00101; A61B 1/00131; A61B 1/00137; A61B 1/00148; A61B 1/012; A61B 1/31; A61B 17/320016; A61B 17/32056; A61B 1/04

(57) ABSTRACT

Systems, methods and devices for performing an endoscopic procedure are provided herein, including an endoscopic plug designed to create a sealed environment within a body cavity using an internal expandable portion which maintains a collapsed configuration during an insertion process or removal process, but which can be expanded upon insertion into the body cavity to secure an internal portion of the endoscopic plug within the body cavity while creating a seal to prevent leakage of gas or fluid out of the body cavity during the endoscopic procedure.

### RELATED APPLICATIONS

This is a continuation of U.S. application Ser. No. 16/929,057, filed Jul. 14, 2020, which is a continuation-in-part of U.S. application Ser. No. 16/775,219, filed Jan. 28, 2020, which is a continuation of U.S. application Ser. No. 14/777,393, filed Sep. 15, 2015, issued as U.S. Pat. No. 10,542,871, which is a National Stage Entry of International Application No. PCT/US2014/030568, filed Mar. 17, 2014, which claims the benefit of the priority of U.S. Provisional Application No. 61/801,427 filed Mar. 15, 2013 and U.S. Provisional Application No. 61/786,520, filed Mar. 15, 2013, which are incorporated herein by reference in entirety.

### FIELD OF THE INVENTION

The embodiments described herein are related to systems, methods and devices for performing endoscopic procedures, and more particularly to a multi-functional plug which creates a sealed environment within a body cavity for performing an endoscopic procedure therein.

### BACKGROUND OF THE INVENTION

Endoscopy is a minimally invasive medical procedure where an endoscope is inserted into a body cavity in order to view the interior of the body cavity. If the body cavity has no orifice to the external environment, an opening must be created through the skin for insertion of the endoscope. If the body cavity has an existing orifice, such as the colon or esophagus, the endoscope can be inserted through the orifice without requiring a separate opening. Once the opening is created, a gas—either air or carbon dioxide—is inserted into the cavity to inflate the body cavity for better viewing by the endoscope. Although endoscopy may be used simply to view the body cavity for diagnostic purposes, one or more tools may be inserted into the body cavity at the same time in order to perform an interventional diagnostic or therapeutic procedure.

Water aided endoscopy (WAE) is a type of endoscopy where the body cavity is filled with water instead of a gas. WAE provides several benefits over gas insufflation, including less distention of the body cavity and image magnification, but also provides significant challenges. While some leakage of air in an endoscopic procedure is expected and does not cause problems, leakage of water from WAE can cause many problems during the procedure, as leaking water may interfere with an individual performing the procedure and must be cleaned up.

### SUMMARY

Embodiments described herein provide systems, methods and devices for performing endoscopic procedures, including an endoscopic plug which is designed to create a sealed environment within the body cavity using an inflatable or expandable portion located on a distal section of the plug.

In one embodiment, an endoscopic plug comprises: a base pad located at a proximal end of the endoscopic plug and configured for positioning adjacent a body cavity opening, wherein the base pad is configured to create a seal between the body cavity opening and an external environment; a shaft extending from the base pad to a distal end of the endoscopic plug and configured for insertion into a body cavity via the body cavity opening; and an expandable portion positioned at the distal end of the endoscopic plug, wherein the expandable portion is configured to expand into an expanded position to securely seal the plug within a body cavity.

In another embodiment, a method of securing an endoscopic plug into a body cavity opening, the method comprising the steps of: inserting a distal end of a shaft of an endoscopic plug through a body cavity opening into a body cavity, wherein the distal end of the shaft includes an expandable portion in a collapsed position; positioning a base pad located at a proximal end of the endoscopic plug in an external environment against the body cavity opening; and expanding the expandable portion into an expanded position within the body cavity to securely seal the endoscopic plug within the body cavity.

In a further embodiment, an endoscopic plug comprises: a base pad located at a proximal end of the endoscopic plug and configured for positioning adjacent a body cavity opening, wherein the base pad is configured to create a seal between the body cavity opening and an external environment; a shaft extending from the base pad to a distal end of the endoscopic plug and configured for insertion into a body cavity via the body cavity opening, wherein the shaft includes an opening to allow passage of a fluid or gas from an external environment to the expandable portion to expand and collapse the expandable portion; and an expandable portion positioned at the distal end of the endoscopic plug, wherein the expandable portion is configured to expand into an expanded position to securely seal the plug within a body cavity.

### BRIEF DESCRIPTION OF THE DRAWINGS

The accompanying drawings, which are incorporated in and constitute a part of the specification, illustrate embodiments of the invention and, together with the description, serve to explain the objects, advantages, and principles of the invention. In the drawings:

FIG. 1 is a perspective view of an endoscopic plug for use in an endoscopic procedure, according to one embodiment of the invention;

FIG. 2 is a horizontal cross-sectional view of the endoscopic plug, according to one embodiment of the invention;

FIG. 3 is a vertical cross-sectional view of the endoscopic plug, according to one embodiment of the invention;

FIG. 4 is an exploded-view of the endoscopic plug, according to one embodiment of the invention;

FIG. 5 is an exploded-view illustration of an endoscopic plug with an expandable sleeve, according to a second embodiment of the invention;

FIG. 6 is a perspective view of a second embodiment of an assembled endoscopic plug with an expandable distal end, according to one embodiment of the invention;

FIG. 7 is a perspective view of the second embodiment of the endoscopic plug that is ready for insertion, according to one embodiment of the invention;

FIG. 8 is a perspective view illustration of a base plug with openings for attaching straps to anchor the plug, according to one embodiment; and

FIG. 9 illustrates a method of performing an endoscopic procedure using the endoscopic plug described herein, according to one embodiment.

### DETAILED DESCRIPTION OF EMBODIMENTS OF THE INVENTION

After reading this description it will become apparent to one skilled in the art how to implement the invention in various alternative embodiments and alternative applications. However, all the various embodiments of the present invention will not be described herein. It is understood that the embodiments presented here are presented by way of an example only, and not limitation. As such, this detailed description of various alternative embodiments should not be construed to limit the scope or breadth of the present invention as set forth below.

The systems, methods and devices described herein provide a more effective tool for performing endoscopic procedures. Embodiments described herein primarily focus on performing a colonoscopy, although the principles are applicable to other endoscopic procedures, both rigid (laparoscopy, thoracoscopy) and flexible.

In one embodiment, a specialized plug is designed for inserting into the rectum so as to create a seal around the periphery of the rectum and prevent liquid from leaking or spilling during a colonoscopy. Additional devices connected with the plug will also operate to create a seal around the periphery of the opening or hold the plug in place so as to prevent any liquid from leaking out. Specific features of the plug are described further below.

The plug is also configured with one or more openings which allow the controlled passage of water, gas and tools through the plug and into the body cavity without allowing liquid to leak through the openings. The various openings may be of different diameters and positioned at different locations on the plug, as will be set forth below. One or more tubes may connect with respective openings in the plug and protrude from an external surface of the plug to connect with devices to deliver liquid, gas or other materials into the body cavity.

Additional embodiments will be described in detail further herein.

### I. Endoscopic Plug

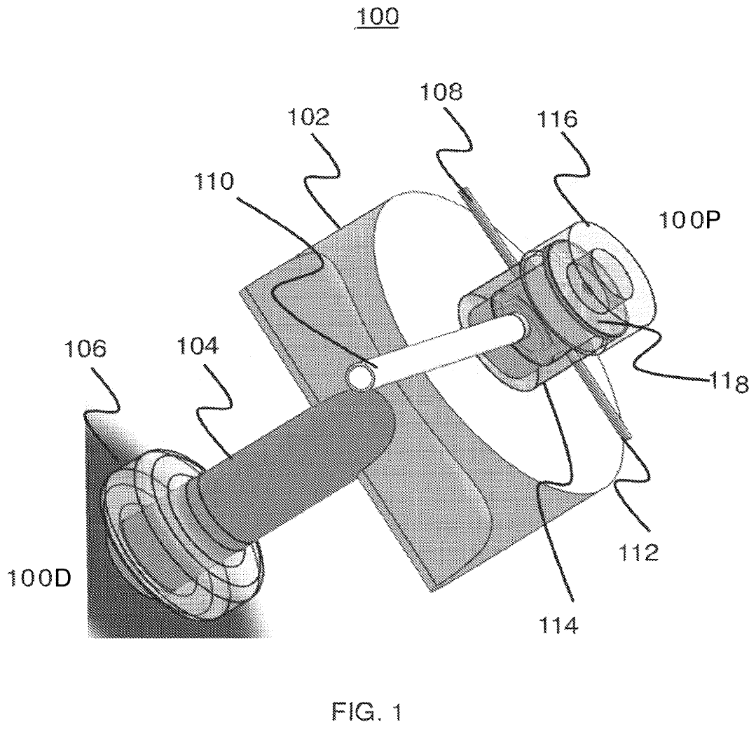

FIGS. 1-4 illustrate one embodiment of an endoscopic plug 100 configured for placement in an opening between a body cavity and an external environment. In a colonoscopy procedure, the plug 100 would be placed in the rectum of a patient. As illustrated in FIG. 1, the plug 100 may include a base pad 102 which is fitted into the opening between the body cavity and the external environment. The distal or interior end 100D of the plug may include a plug shaft 104 protruding into the body cavity and a balloon mechanism 106 used to create an additional seal, as will be described further below. The proximal or exterior end 100P of the plug 100 may include a conduit 114 which provides openings with one or more tubes connected with the openings to other mechanisms which deliver gas and/or liquid through the device and into the body cavity or which remove liquid from the body cavity. A central core of the plug 100 is sealably closed with a seal 118 positioned on the proximal side 100P of the base pad 102 to expand and contract in response to the insertion or removal of a tool, so as to create a water-tight environment. A seal cap 116 may be positioned over the seal 118 to hold the seal 118 in place and prevent liquid from spilling out through a scope channel 102C where a tool or optical device is passed into the body cavity.

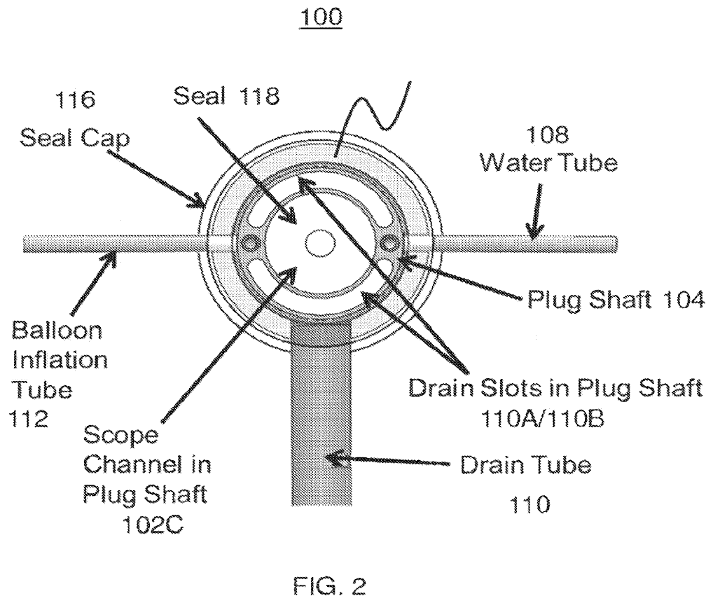

In one embodiment, the plug 100 may include one or more openings designed for input and output of a fluid used to maintain a constant pressure of liquid within the body cavity. In FIG. 2, an input opening in the outer circumference of the shaft may be connected to an input water tube 108 which feeds liquid into the body cavity from a pump. An output tube, or drain tube 110, may be connected with one or more drain slots 110A and 110B, or output openings, to drain liquid from the body cavity. The input water tube 108 and the output drain tube 110 allow a user performing the medical procedure to regulate the pressure inside the body cavity and adjust the pressure if it is too high or low. A pressure regulator or relief valve (not shown) could also be included in the drain line to further control the pressure within the body cavity.

The plug may also include one or more openings for insertion of an endoscopic tool via the scope channel 102C in the plug shaft in FIG. 2. The endoscopic tool may be an endoscope with an image capture device that is inserted into the body cavity for capturing images of the body cavity and transmitting the images to a display device in the external environment. The opening of the scope channel 102C may be expandable and re-sealable using the seal 118 so that the tools may be completely removed from the plug without causing liquid to leak out of the body cavity through the tool opening and expanded to permit a larger tool to pass through. In one embodiment, the input opening for the water tube 108 may be eliminated and integrated into an endoscope tool for pushing water along the shaft of the tool deep into the body cavity. The ability to eject liquid on or near a distal end of the endoscope may prove useful when the user needs to flush away debris or manipulate tissue within the body cavity.

In one embodiment, the drain slots 110A and 110B and the scope channel opening 102C may be a single opening in order to allow for a larger opening which can accommodate larger tools or multiple tools. Liquid exiting through the centralized output opening and scope channel may be collected in a reservoir disposed on the outside surface of the plug, where the collected liquid is then fed into the drain tube.

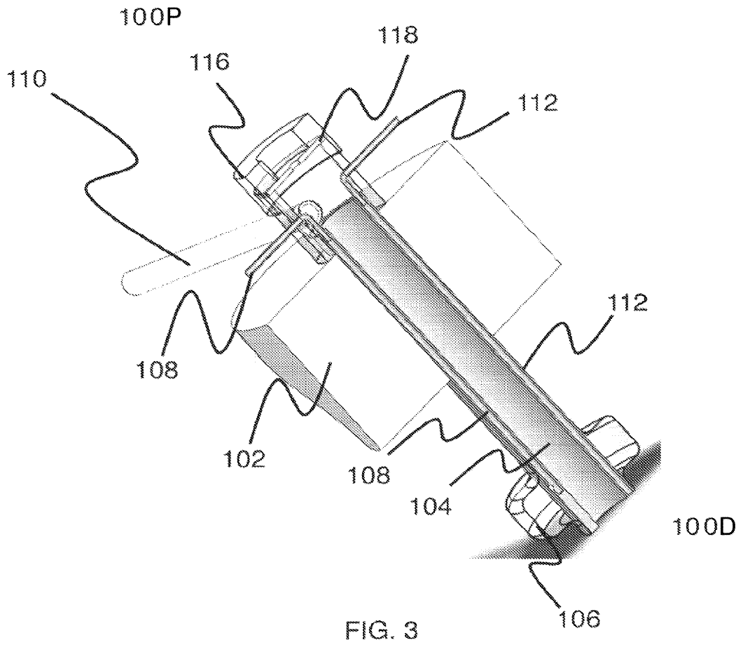

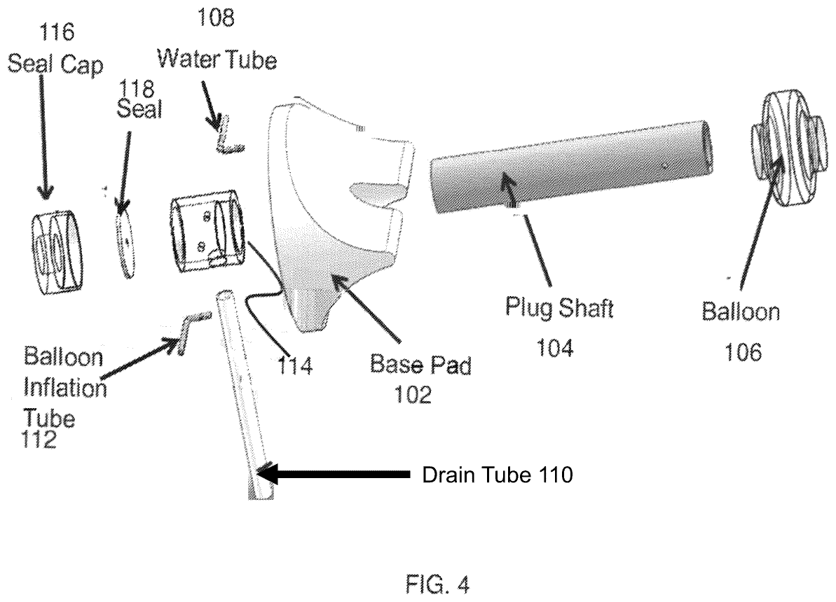

In one embodiment, the plug 100 is configured with an inflatable balloon sealing mechanism 106 which is located on the distal end 100D of the plug, as shown in FIG. 1, FIG. 3 and FIG. 4. The balloon 106 may be transparent and provide a better view of the anatomical features surrounding the shaft 104, particularly in comparison with a solid sealing object. An opening in the plug would connect with a balloon inflation tube 112 on the proximal end 100P of the plug so that air passing into the body cavity through the opening would inflate the balloon 106 once the plug is inserted into the opening in the body cavity. In one embodiment, the balloon 106 could also be inflated with fluid. The inflated balloon 106 is expanded to a diameter larger than the plug 100 so that it is more tightly positioned against the inner wall of the natural or artificially-created orifice than the plug 100. The inflated balloon 106 is connected with the plug 100 via the plug shaft 104 and therefore maintains the plug in a more tightly sealed position than the plug does on its own, while also preventing leaks with a pressurized seal. Even though the balloon 106 is shown in a cylindrical shape, it is envisioned that the shape of the balloon can be configured in a different shape to better seal with the internal surface of the body cavity.

In another embodiment, a retention disc (not shown) may be positioned on the proximal end 100P of the plug 100 in the external environment in order to hold the plug 100 in place from the proximal end 100P. A flexible retention disc may be positioned against the skin of the patient over the top of the plug and be configured to tighten around the skin to hold the plug 100 in place within the body cavity opening. The retention disc may be used separately or in conjunction with the inflatable balloon 106 in order to create a leak-proof seal around the plug.

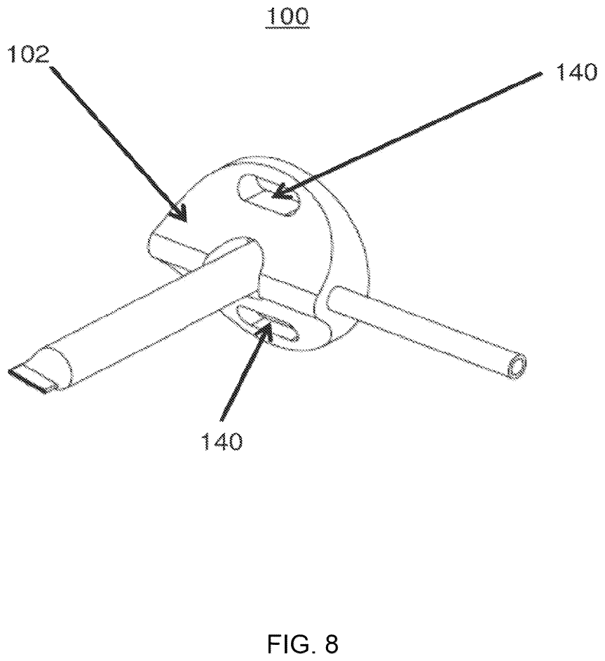

In a further embodiment of the plug, the base pad 102 may be held against the rectal opening using straps attached to an external table, or the base pad 102 may be incorporated into an article of clothing worn by the patient during the examination. An illustration of the base pad 102 with openings 140 for attaching the straps is illustrated in FIG. 8. By providing an external mechanism for holding the base pad in place, the plug shaft, balloon and internal expanded seal may be eliminated as a secondary sealing mechanism, which will then allow the physician to view areas of the rectum and colon immediately surrounding the base pad. In this embodiment, a thin membrane (such as latex) may be provided as the sleeve 120, along with a windsock-type seal 120A, so that tools entering the colon can immediately be accessible within the colon and allow viewing and interaction with the entire colon.

The plug shaft 104 illustrated in FIG. 4 may also operate as a windsock sealing mechanism which collapses when a tool is removed therefrom, maintaining a seal between the body cavity and the external environment.

### II. Pressure Valve

In one embodiment, a pressure valve (not shown) may be used in order to maintain the pressure of the liquid within the body cavity. In one embodiment, the pressure valve may be positioned on the drain tube 110 of the plug to regulate the liquid pressure and maintain an optimal pressure within the body cavity that is advantageous for performing operative procedures, but which does not cause discomfort to the patient or increase to the point that it causes leaks. The valve may be configured to release upon exceeding a certain desired pressure.

In an alternate embodiment, the pressure valve may be positioned within the input opening or within the input water tube 108 and be configured to constrict, fully close or fully open depending on the pressure of the fluid being input into the body cavity.

### III. Distal Sealing Mechanism

In one embodiment, a distal sealing mechanism may be provided which is inserted through the plug and positioned at one end of a body cavity distal from the plug 100 (such as the proximal, or upstream end of the colon near the cecum) in order to create a completely sealed environment within a larger body cavity which can be filled with liquid for examination. As previously illustrated in FIG. 1, FIG. 3 and FIG. 4, the distal sealing mechanism may be an inflatable balloon 106 which inflates after being inserted into the body cavity (although the balloon would be positioned much further down into the body cavity than that illustrated in FIG. 1, FIG. 3 and FIG. 4). The balloon may require a separate input opening and balloon inflation tube 112 in the plug that passes a gas or fluid through the plug (as shown in FIG. 3), through the body cavity and into the balloon at the distal end. Once the balloon 106 is inflated, liquid can be pumped into the artificially created cavity for examination of a specific area.

In another embodiment, a flexible conical-shaped plastic surface like the ones used in percutaneous endoscopic gastrostomy (“PEG”) tubes may be used, which contracts for insertion, expands after insertion and tightens as pressure from inside the body cavity pushes on the object.

### IV. Alternative Plug Designs

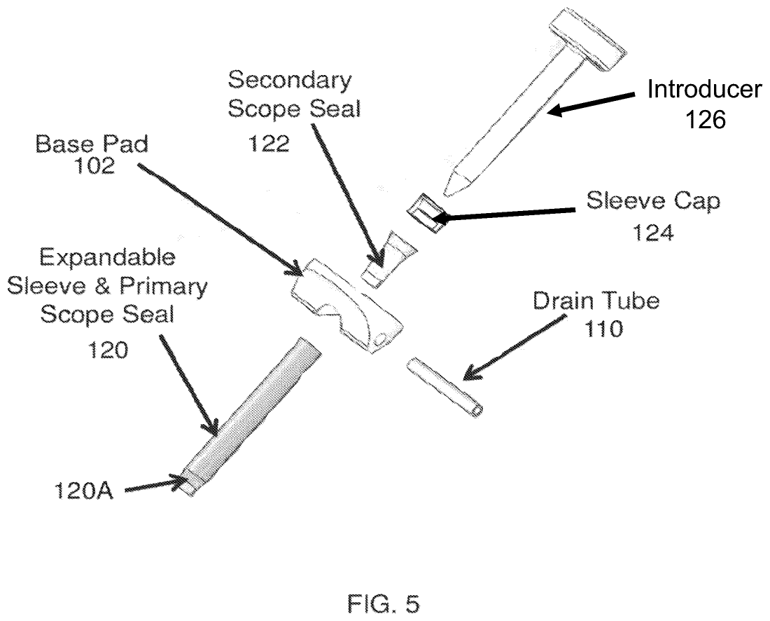

FIGS. 5-7 illustrate an alternative plug design which may also be used for endoscopy. FIG. 5 is an exploded view illustration of the alternative plug design, which features an expandable sleeve 120 and primary scope seal 120A, according to a second embodiment of the invention. This alternative plug design does not have a separate water input tube, as water will be input through the primary endoscope opening itself. FIG. 5 also illustrates the other components, such as a secondary scope seal 122 and a sleeve cap 124. The base pad 102 also incorporates a cylindrical drain tube opening to allow fluid pressurized inside the colon to easily and more safely drain out through the drain tube opening, which is then connected with the drain tube. In one embodiment, two “duck bill-style” seals are used at a proximal end and a distal end of the sleeve 120, although only one seal could be used in order to reduce the sleeve's crossing profile for insertion. The distal seal 120A also serves to reduce the profile of the front end of the sleeve.

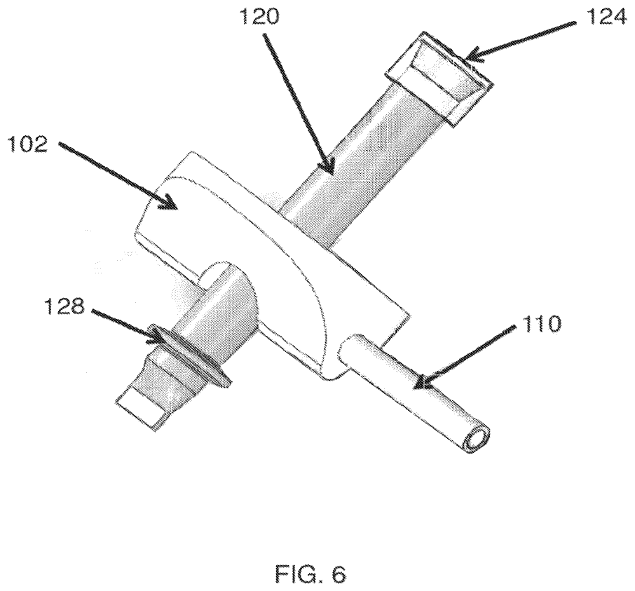

In this embodiment, a distal end 128 of the expandable sleeve 120 (away from the base pad where the primary scope seal 120A is located) is capable of expanding in a lateral direction around the circumference of the sleeve, as shown in the perspective view illustration of the assembled alternative plug design in FIG. 6. This expandable sleeve 120 provides for the expandable distal end 128 to fit against the interior of the rectal opening. The base pad 102 is positioned to slide onto the outer diameter of the expandable sleeve 120 and slides down on the expandable sleeve until it fits against the outer rectal opening. In other words, when the expanded distal end 128 of the expandable sleeve 120 and the base pad 102 are pulled together, it creates a sealed, locked position around the rectal opening. The sleeve cap 124 may be positioned over the expandable sleeve 120.

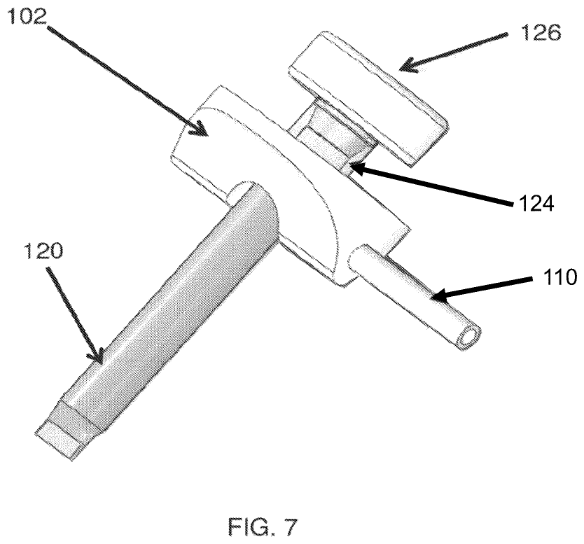

FIG. 7 is a perspective view of the second embodiment of the endoscopic plug in an un-sealed position, according to one embodiment of the invention. The movement of the base pad 102 along the sleeve 120 can be illustrated by comparing the location of the base pad between FIG. 6 and FIG. 7, since the base pad 102 is at the proximal end of the plug device.

A method of inserting the plug and sealing it around the rectal opening will now be described. Once the plug has been inserted into the colon, an introducer is removed and the distal end of the expandable sleeve expands and locks (either mechanically or automatically). The sleeve may then be pulled back to seat against the interior rectal wall opening to form an interior portion of the seal. The base pad is then moved forward (toward the rectal opening) while the sleeve is held in place, allowing the base pad to be fitted up against the external rectal opening; thereby fixing the plug in place from both sides. The introducer is then removed, which frees up the sleeve for the passage of the colonoscope

### V. Tools

In one embodiment, a plurality of endoscopic tools may be configured for insertion through the scope channel 102C of the plug. Endoscopic tools may use cautery (coagulation forceps and probes, snares, knives), enable mechanical cutting (scissors), tissue closure (clips, loops, clamps, stiches), tissue capture (baskets, forceps) and tissue stretching (dilation balloons and bougies). Since the endoscopic tools are operating completely in a liquid environment, some of the tools may require more power in order to achieve a desired coagulation effect or displace the heavier liquid in the body cavity. Other tools may need to be weighted in order to be able to move more easily through the dense liquid.

Other tools may be made more powerful as a result of being in liquid, such as tools which normally produce a great deal of heat. The liquid would more easily dissipate the heat from the tool, allowing the tool to use more power without risking damage or discomfort to the patient. Tools which use electricity or which produce ablative heat may also need to be specifically configured to operate within a liquid environment.

### VI. Image Processing

In one embodiment, the systems and methods for performing an endoscopic procedure include applying image processing algorithms for improving the clarity and quality of images captured by the endoscope. Images taken in a liquid environment may produce unique artifacts and present quality issues not found with images captured in a gas medium. In one embodiment, real-time image enhancement may be performed to improve the clarity of the video images produced by the endoscope and remove artifacts caused by pollutants or other contaminants within the liquid. In one embodiment, an image and video enhancement processor known as the Clarity™ System (Zmed, Inc., San Diego, CA) may be used.

### VII. Methods of Use

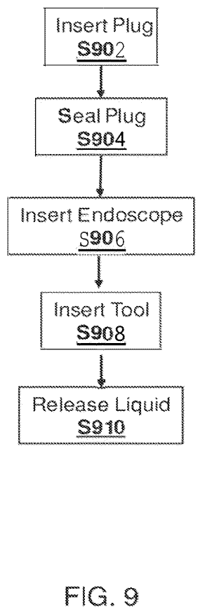

A method for performing an endoscopic procedure with the devices described herein is illustrated in FIG. 9. One step includes inserting a plug into an opening between a body cavity and an external environment (S902); sealing the plug with at least one sealing mechanism (such as a balloon) (S904); and inserting at least one endoscope through an opening in the plug (S906). Additional steps may include inserting at least one tool through an opening in the plug (S908); and releasing a quantity of the liquid from the body cavity through an output opening in the plug (S910).

Methods of manufacturing the endoscopic plug device described herein may also be provided for, based on the steps of assembling the parts of the plug and opening along with the connected tubes, seals, shafts, etc.

What is claimed is:

1. An endoscopic plug comprising:
a base pad located at a proximal end of the endoscopic plug and configured for positioning adjacent a body cavity opening, wherein the base pad is configured to create a seal between the body cavity opening and an external environment;
a shaft extending from the base pad to a distal end of the endoscopic plug and configured for insertion into a body cavity via the body cavity opening; and
an expandable portion positioned at the distal end of the endoscopic plug, wherein the expandable portion is configured to expand into an expanded position to securely seal the plug within a body cavity.

2. The endoscopic plug of claim 1, wherein the expandable portion is configured to collapse into a collapsed position for insertion and removal of the shaft from the body cavity.

3. The endoscopic plug of claim 1, wherein the expandable portion is an inflatable balloon positioned around an outer circumference of the shaft.

4. The endoscopic plug of claim 1, wherein the shaft includes a scope channel opening for insertion of an endoscopic tool into the body cavity.

5. The endoscopic plug of claim 4, wherein the scope channel opening includes a seal preventing a fluid or a gas from leaking from the body cavity to the external environment.

6. The endoscopic plug of claim 1, wherein the shaft includes an opening to allow passage of a fluid or gas from an external environment to the expandable portion to expand and collapse the expandable portion.

7. The endoscopic plug of claim 6, wherein the opening is integrated into an outer circumference of the shaft.

8. A method of securing an endoscopic plug into a body cavity opening, the method comprising the steps of:
inserting a distal end of a shaft of an endoscopic plug through a body cavity opening into a body cavity, wherein the distal end of the shaft includes an expandable portion in a collapsed position;
positioning a base pad located at a proximal end of the endoscopic plug in an external environment against the body cavity opening; and
expanding the expandable portion into an expanded position within the body cavity to securely seal the endoscopic plug within the body cavity.

9. The method of claim 8, further comprising collapsing the expandable portion into a collapsed position for removal of the shaft from the body cavity.

10. The method of claim 8, wherein the expandable portion is an inflatable balloon positioned around an outer circumference of the shaft that is inflated into the expandable position.

11. The method of claim 10, further comprising inflating the inflatable balloon with air.

12. The method of claim 11, further comprising inflating the inflatable balloon via an opening in the shaft which connects the inflatable balloon with an external environment.

13. The method of claim 8, wherein the positioning of the base pad against the body cavity opening creates a seal between the body cavity opening and an external environment.

14. The method of claim 9, further comprising inserting an endoscopic tool into the body cavity through a scope channel opening in the shaft.

15. An endoscopic plug comprising:
a base pad located at a proximal end of the endoscopic plug and configured for positioning adjacent a body cavity opening, wherein the base pad is configured to create a seal between the body cavity opening and an external environment;
a shaft extending from the base pad to a distal end of the endoscopic plug and configured for insertion into a body cavity via the body cavity opening, wherein the shaft includes an opening to allow passage of a fluid or gas from an external environment to the expandable portion to expand and collapse the expandable portion; and
an expandable portion positioned at the distal end of the endoscopic plug, wherein the expandable portion is configured to expand into an expanded position to securely seal the plug within a body cavity.

16. The endoscopic plug of claim 15, wherein the expandable portion is configured to collapse into a collapsed position for insertion and removal of the shaft from the body cavity.

17. The endoscopic plug of claim 15, wherein the expandable portion is an inflatable balloon positioned around an outer circumference of the shaft.
